(19) United States
(12) Patent Application Publication
(10) Pub. No.: US 2017/0224245 A1
(43) Pub. Date: Aug. 10, 2017

(54) SYSTEM FOR DIAGNOSING AND TREATING NEUROLOGICAL AND PSYCHOLOGICAL DISORDERS

(71) Applicants: CHIUN-FAN CHEN, Brookline, MA (US); LUNG-CHANG LIN, Kaohsiung (TW)

(72) Inventors: CHIUN-FAN CHEN, Brookline, MA (US); LUNG-CHANG LIN, Kaohsiung (TW)

(21) Appl. No.: 15/019,620

(22) Filed: Feb. 9, 2016

(51) Int. Cl. A61B 5/0476; A61N 1/36; A61B 5/11; A61B 5/04

(52) U.S. Cl. CPC A61B 5/0476; A61B 5/04012; A61N 1/36025; A61B 5/11

(57) ABSTRACT

A medical system including electrodes for acquiring electroencephalography (EEG) signals, amplifiers to amplify the EEG signals, an analog-to-digital converter to generate digitalized EEG data from the EEG signals, and a processor for processing the digitalized EEG data to generate a indication of neurological or psychological symptom based on a combination selected among spectral, temporal, and spatial features of the digitalized EEG data. The medical system further including a transcranial electrical stimulator that regulates stimulation frequency, intensity, or polarity according to the indication.

### RELATED APPLICATIONS

This application claims the benefit of U.S. Provisional Application Ser. No. 62/114,547, filed Feb. 10, 2016, the entire disclosure of which is herein incorporated by reference.

### BACKGROUND OF THE INVENTION

1. Field of the Invention

The invention relates to a medical system and, more particularly, to a medical system that provides a diagnosis for neurological or psychological disorders and treats the disorders based upon the diagnosis.

2. Description of the Related Art

Modern advances in science and technology allow us to know a lot more about the human brain than we did in the past. Innovative and cutting-edge devices help today's medical professionals and researchers to conduct in-depth observations of different physiological characteristics within the brain. Nonetheless, such observations altogether, have neither been fully evaluated to generate a diagnosis for neurological or psychological disorders nor been utilized to serve as an indication of how transcranial electrical stimulators could be controlled to improve symptoms of the disorders.

### BRIEF SUMMARY OF THE INVENTION

One of the objects of the present invention is to implement a medical system that is capable of providing an indication of neurological or psychological disorders and treating the disorders based upon the indication.

According to one embodiment of the invention, a medical system is disclosed. The medical system includes electrodes for acquiring electroencephalography (EEG) signals, amplifiers coupled to the electrodes to amplify EEG signals, an analog-to-digital converter coupled to the amplifiers to generate digitalized EEG data from the EEG signals, and a processor for processing the digitalized EEG data to generate an indication of neurological or psychological symptom based on a combination selected among spectral, temporal, and spatial features of the digitalized EEG data.

Moreover, the system may further include a transcranial electrical stimulator that regulates stimulation frequency, intensity, or polarity according to the indication.

### BRIEF DESCRIPTION OF THE DRAWINGS

FIG. 1 shows a schematic diagram of a medical system illustrated according to an embodiment of the present invention.

FIG. 2 shows how a transcranial electrical stimulator can be incorporated into an embodiment of the medical system.

### DETAILED DESCRIPTION OF THE INVENTION

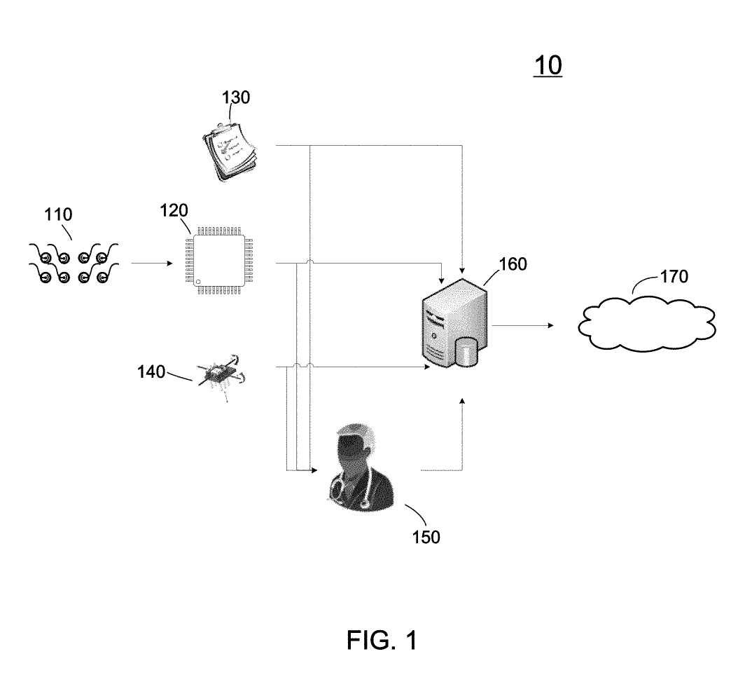

FIG. 1 shows a schematic diagram of a medical system 10 illustrated according to an embodiment of the present invention. The medical system 10 includes electrodes 110 for acquiring electroencephalography (EEG) signals, amplifiers (not shown in the figure) coupled to the electrodes to amplify the EEG signals, an analog-to-digital converter (ADC, not shown in the figure) coupled to the amplifiers to generate digitalized EEG data from the EEG signals, and a processor 120 for processing the digitalized EEG data to generate a first indication of neurological or psychological symptom based on a combination selected among spectral, temporal, and spatial features of the digitalized EEG data. In a preferred embodiment, the spectral feature is derived from mathematical manipulation of power spectral densities in different frequency bands of the EEG, the temporal feature is derived from computed EEG changes before and after an event or task performance, and the spatial feature is derived from coherence or correlation of EEG channels that correspond to brain connectivity. It should be noted that the amplifier and ADC can be discrete electronic components or be integrated with the processor. Detailed description of the implementation is well known and is therefore omitted herein for brevity.

In a preferred embodiment, the medical system 10 may also include an accelerometer 140 and/or a gyroscope to generate a second indication that represents the motion and displacement of the human body, preferentially attached to the extremity of interest.

In another preferred embodiment, the medical system 10 may include a database 160, wherein the database 160 collects scores (e.g., DSM-IV or -V evaluation scores) of neurological or psychological evaluation questionnaires 130. A physician 150 may be involved in determining the applicability of the first and second indications and the evaluation questionnaires 130 to generate diagnoses information. The database 130 can upload the diagnoses information to a cloud storage 170 to make it available to hospitals and clinics in other regions of the world.

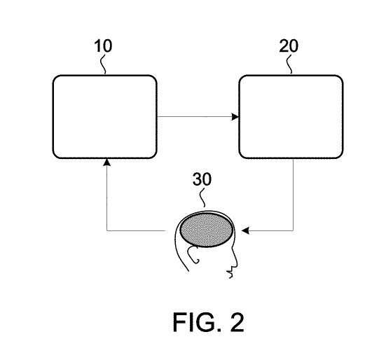

In yet another preferred embodiment, as illustrated in FIG. 2, the medical system 10 may further include a transcranial electrical stimulator 20. The transcranial electrical stimulator 20 regulates stimulation frequency, intensity, or polarity according to the first indication generated by the processor. In another embodiment, the transcranial electrical stimulator 20 can regulate stimulation frequency, intensity, or polarity according to both the first and second indications. It should be noted that the transcranial electrical stimulator 20 could be a transcranial direct current stimulator, a transcranial alternating current stimulator, or a transcranial pulsed current stimulator, depending on which is more appropriate for a certain clinical setting.

While the invention has been described by way of examples and in terms of the preferred embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A medical system, comprising:
a plurality of electrodes, for acquiring electroencephalography (EEG) signals;
a plurality of amplifiers, coupled to the electrodes to amplify the EEG signals;
an analog-to-digital converter, coupled to the amplifiers to generate digitalized EEG data from the EEG signals; and
a processor, for processing the digitalized EEG data to generate a first indication of neurological or psychological symptom based on a combination selected among spectral, temporal, and spatial features of the digitalized EEG data.

2. The system of claim 1, further comprising a transcranial electrical stimulator, wherein the transcranial electrical stimulator regulates stimulation frequency, intensity, or polarity according to the first indication.

3. The system of claim 1, further comprising a transcranial electrical stimulator, wherein the transcranial electrical stimulator is a transcranial direct current stimulator, a transcranial alternating current stimulator, or a transcranial pulsed current stimulator.

4. The system of claim 1, further comprising an accelerometer and/or a gyroscope to generate a second indication that represents the motion and displacement of the human body.

5. The system of claim 4, further comprising a transcranial electrical stimulator, wherein the transcranial electrical stimulator regulates stimulation frequency, intensity, or polarity according to the first and second indications.

6. The system of claim 1, wherein the spectral feature is derived from mathematical manipulation of power spectral densities in different frequency bands of the EEG.

7. The system of claim 1, wherein the temporal feature is derived from computed EEG changes before and after an event or task performance.

8. The system of claim 1, wherein the spatial feature is derived from coherence or correlation of EEG that correspond to brain connectivity.

9. The system of claim 1, further comprising a database, wherein the database collects a plurality of scores of neurological or psychological evaluation questionnaires.

10. The system of claim 9, wherein the database further collects diagnoses information based on the scores and at least the first or second indication.

11. The system of claim 10, wherein the database uploads the diagnoses information to a cloud storage to make it available to hospitals and clinics in other regions.
